(19) United States
(12) Patent Application Publication
(10) Pub. No.: US 2014/0274928 A1
(43) Pub. Date: Sep. 18, 2014

(54) IDENTIFYING AND TREATING PATIENTS WITH CASTRATE RESISTANT PROSTATE CANCER

(71) Applicants: Robert A. Dowling, Fort Worth, TX (US); Tracey Gray, Springfield, MO (US); Richard Kroutil, Springfield, MO (US); Kelly Huang, Austin, TX (US)

(72) Inventors: Robert A. Dowling, Fort Worth, TX (US); Tracey Gray, Springfield, MO (US); Richard Kroutil, Springfield, MO (US); Kelly Huang, Austin, TX (US)

(73) Assignee: HEALTHTRONICS INFORMATION TECHNOLOGY SOLUTIONS, INC., AUSTIN, TX (US)

(21) Appl. No.: 14/213,913

(22) Filed: Mar. 14, 2014

Related U.S. Application Data
(60) Provisional application No. 61/799,005, filed on Mar. 15, 2013.

(51) Int. Cl. G06F 19/00; A61B 18/02

(52) U.S. Cl. CPC G06F 19/345; A61B 18/02
USPC 514/34; 705/3; 606/20

(57) ABSTRACT

This disclosure relates generally to the field of prostate cancer and, in particular, methods of identifying and treating patients having castrate resistant prostate cancer.

### CROSS-REFERENCE TO RELATED APPLICATIONS

This application claims the benefit of U.S. Provisional Application No. 61/799,005, filed Mar. 15, 2013, which is hereby incorporated by reference in its entirety.

### TECHNICAL FIELD

This disclosure relates generally to the field of prostate cancer and, in particular, methods of identifying and treating patients having castrate resistant prostate cancer.

### BACKGROUND

Prostate cancer is the most common malignancy for older men and is a major cause of death for that population. Until recently, it was believed that reduction of testosterone was a key component in treating patients diagnosed with prostate cancer. However, a large number of patients having prostate cancer do not respond to reduction of testosterone levels instigated by luteinizing hormone releasing hormone (“LHRH”) agonists or antagonists, and were thereby dubbed as having “hormone resistant” or “castrate resistant” prostate cancer. Only about half of patients having castrate resistant prostate cancer (“CRPC”) respond to hormonal treatments. It is currently recognized that LHRH agonists or antagonists do not completely reduce circulating testosterone levels because sources other than the testes can synthesize testosterone, including the adrenal gland and the prostate tumor cells themselves.

Diagnosis of a patient with CRPC confers prognostic implications and candidacy for advanced treatments. There is no single marker in claims data (ICD-9 or other nomenclature) or clinical data to identify patients with CRPC. While there is no single endorsed definition for this state, there is general consensus that patients must be on continuous and effective androgen suppression therapy, and show evidence of progression. Physicians and others need a method for easily and accurately identifying these patients.

Accordingly, disclosed herein, inter alia, are methods to identify patients with CRPC, the date this status was achieved, and courses of treatment for a patient identified as having CRPC.

### SUMMARY OF THE INVENTION

In some embodiments, methods of treating a patient with castrate resistant prostate cancer (CRPC) are provided. In some embodiments, the method comprises performing a CRPC search method to identify a patient with castrate resistant prostate cancer and treating the patient identified with CRPC. In some embodiments, the CPRC search method is performed according to a method provided herein.

In some embodiments, a method of treating a patient with castrate resistant prostate cancer is provided. In some embodiments, the method comprises performing a CRPC search method to identify a patient with castrate resistant prostate cancer on a computer system programmed with software configured to perform the CRPC search method; and treating the patient identified with CRPC. In some embodiments, the CPRC search method is performed according to a method described herein.

In some embodiments, data processing systems for identifying a patient with castrate resistant prostate cancer are provided. In some embodiments, the data processing system comprises a computer processor for processing data, wherein the computer processor accesses an electronic health records database; and the computer processor executes a CRPC search method with the accessed data to identify a patient with CRPC. In some embodiments, the CRPC search method to identify the patient with CRPC executed by the computer processor comprises a method described herein.

In some embodiments, methods for identifying for identifying a patient with castrate resistant prostate cancer are provided. In some embodiments, the method comprises a data processing system that comprises a computer processor for processing data, wherein the computer processor accesses data in an electronic health records database where the computer processor executes a CRPC search method with the accessed data to identify a patient with CRPC. In some embodiments, the CRPC method executed is a method described herein.

In some embodiments, methods of identifying a patient with castrate resistant prostate cancer are provided, wherein the method comprises a method described herein.

In some embodiments, methods of identifying a patient with metastatic prostate cancer are provided, wherein the method comprises a method described herein.

### DESCRIPTION

Embodiments disclosed herein are directed to identifying patients included in a medical health records database, which may or may not be electronic, as having castrate resistant prostate cancer (“CRPC”). Thus, the methods disclosed herein may be performed on a computing system or may be performed manually by physically reviewing the patient medical records in a database. Once identified, these patients can be prescribed or administered a course of therapy in order to treat the CRPC. As used herein, CRPC includes prostate tumors or prostate cancer cells that are restricted to the prostate, and prostate tumors or prostate cancer cells that are not restricted to the prostate (such as metastatic castrate resistant prostate cancer).

As used herein, a patient with CRPC is a patient, inter alia, while on GnRH drugs, who experiences at least three consecutive rises in Prostate Specific Antigen (“PSA”) levels and during that time has a testosterone measure of less than or equal to 50 ng/ml. Even if the PSA values decrease or stay the same before or after the three consecutive rises, the patient remains classified as potentially having CPRC. Once a patient is identified as potentially having CRPC, the other reviews, interrogations, or method steps described herein will further determine the patient's likelihood of having CRPC.

As used herein, “treating” CRPC means the application of any suitable therapy for delaying, reducing or ameliorating some or all of the symptoms or conditions associated with the CRPC, including the physical removal of cancerous tissue or cells (e.g., partial or full prostatectomy), ablation of cancerous tissue or cells, for example, by heat, cold (e.g., cryogenic ablation), radiation, radiofrequency ablation, laser ablation, and the like, and pharmaceutical intervention, including administration of therapeutically effective amounts of cytotoxins such as Cabazitaxel (Jevtana®), Mitoxantrone (Novantrone®), Estramustine (Emcyt®), Doxorubicin (Adriamycin®), Etoposide (VP-16), Vinblastine (Velban®), Paclitaxel (Taxol®), Carboplatin (Paraplatin®), Vinorelbine (Navelbine®), and/or administration of therapeutically effective amounts of CYP17 inhibitors or androgen receptor modulators (for example, androgen receptor antagonist).

In some embodiments, an aggregated database of patient medical records is analyzed to identify relevant data elements to produce a longitudinal “timeline” for every patient in the database. Once a longitudinal timeline for every patient in the database is generated, a CRPC search method is then executed against this timeline or staged data to identify patients that meet the following criteria: alive, diagnosed with prostate cancer, continuously on hormone therapy (a sub method is run to define continuous therapy), and rising PSA levels (sub method run to identify significant and sequential rises concurrent with continuous hormone therapy). Generation of the timeline for every patient in the database and executing the CRPC search method (including the sub methods) comprise the CRPC Search Method, which includes those disclosed herein.

As an option, an additional step in the CRPC Search Method can be to include the effectiveness of the LHRH agonists and/or antagonist therapy. The effectiveness of the therapy is determined by requiring a castrate level of testosterone to be present concurrent with the other above-identified criteria.

The search methods include those described herein and can be used to: (i) identify and treat patients having CRPC, (ii) identify and treat patients having metastatic prostate cancer, and/or (iii) determine the initial diagnosis date for prostate cancer.

In some embodiments, a method of treating a patient with CRPC is provided. In some embodiments, the method comprises performing the CRPC Search Method as described herein, to identify patients with CRPC; and then treating the patient identified as having CRPC. In some embodiments, the CRPC is metastatic castrate resistant prostate cancer.

In some embodiments, a method of treating a patient with CRPC is provided. In some embodiments, the method comprises performing the CRPC Search Method as described herein on a computer system programmed with software configured to perform the CRPC Search Method on a database as described herein to identify patients with CRPC; and treating the patients identified as having CRPC. In some embodiments, the CRPC is metastatic CRPC.

In some embodiments, a data processing system for identifying a patient with CRPC is provided. In some embodiments, the data processing system comprises a computer processor for processing data, wherein the computer processor is configured to access an electronic health records database (e.g. medical health records database); and wherein the computer processor is configured to execute the CRPC Search Method according to a method described herein on data/records included in the electronic health records database to identify patients with CRPC. In some embodiments, the data processing system identifies a patient as having metastatic castrate resistant prostate cancer. In some embodiments, a patient identified as having CRPC or metastatic CRPC is treated for said disease.

In some embodiments, the treatment of a patient identified with CRPC or metastatic CRPC according to the methods described herein is selected from the group consisting of administering a CYP17 inhibitor, administering an androgen receptor modulator, administering a cytotoxin, performing a partial prostatectomy, performing a full prostatectomy, and ablation of prostate cancer tissue or cells.

In some embodiments, a method for identifying a patient with CRPC is provided. In some embodiments, the method comprises providing a data processing system comprising a computer processor for processing data, wherein the computer processor is configured to access an electronic medical health records database; and wherein the computer processor is configured to execute the CRPC Search Method disclosed herein on data/records included in the electronic health records database to identify patients with CRPC.

In some embodiments, a method of identifying a patient with CRPC is provided. In some embodiments, the method comprises manually performing the CRPC Search Method as disclosed herein to identify patients with CRPC.

In some embodiments, the CRPC Search Methods described herein comprise reviewing a medical health records database.

In some embodiments, the CRPC Search Method comprises creating a CRPC table with the following fields:
a) an External Patient ID field;
b) one or more Raw Text fields;
c) one or more Procedure Date fields;
d) one or more Value fields;
e) one or more Injection fields;
f) one or more Injection Dose fields;
g) an Initial Injection field set to zero;
h) a Testosterone Flag field set to zero;
i) a PSA Flag field set to zero;
j) a First ASTRO Rise Date field;
k) a ASTRO Rise Date field,
l) a PSA Rise Count field set to zero;
m) a PSA Fall Count filed set to zero;
n) a PSA Last Trend field;
o) a CRPC No Testosterone field set to zero; and
p) a CRPC Testosterone field set to zero.

In some embodiments, a single CRPC Table is created for the medical health records or database that is being searched to identify CRPC patients. The format of the CRPC Table can be, for example, a table where the above-discussed fields correspond to columns in the table and the rows correspond to the patients in the database that have been identified as potentially having CRPC. However, the format of the CRPC Table is not critical and other formats than those described herein can also be used to arrange the information for analysis.

In some embodiments, in order to populate the CRPC Table, a review of the patient medical health records included in the database is performed to identify patients that meet certain criteria, which identifies the patient as potentially having CRPC. For example, a medical health record can be populated with raw data elements, processed data elements, and derived data elements. A non-limiting example of a raw data element is an external patient identification (External Patient ID). The raw data elements can be copied into the appropriate field in the CRPC Table. A processed data element is, for example, information that is processed or generated from the raw data in order to populate different fields in the CRPC Table. For example, the Injection field in the table above can be populated based upon raw data that is processed by a computer. The computer processor can be configured (with software, for example) to search patient medical health records to identify the drug or drugs that a patient has taken or has been administered. This raw data can then be processed to populate the Injection field. Certain fields are populated by having them derived, or calculated based upon other data elements. For example, information for the ASTRO Rise Date, CRPC No Testosterone, and CRPC Testosterone fields are derived from other data elements as discussed herein. For example, the ASTRO Rise Date field will be populated with a specific date based upon whether certain treatments have been administered/prescribed and whether a patient's PSA levels have increased over a certain period of time. Other factors can also be used to determine the date. However, this field is derived based upon other data included in the patient's medical health record. Other examples of how the different fields are populated are discussed herein. The table described above is a non-limiting example and, in some embodiments, one or more of the fields are not included or not populated by the search method.

Once a patient from a database is identified as potentially having CRPC, the CRPC Table is populated with that patient's data in accordance with the following. A patient can be identified as a potential CRPC patient because they have had a GnRH procedure as discussed below. For example, in some embodiments, the CRPC Table is populated with data from a patient's medical health records, which may or may not be in electronic form, if that patient's health records include a CPT code defined as GnRH or a procedure that is indicative of GnRH. In some embodiments, a patient is identified as having a CPT code defined as GnRH when one or more of the following CPT codes in Table 1 below are found in the patient's medical health records.

TABLE 1
CPT Codes and Standard CPT Descriptions

| CPT Code | Standard CPT Procedure Description |
|---|---|
| 11981 | INSERT DRUG IMPLANT DEVICE |
| 11983 | REMOVE/INSERT DRUG IMPLANT |
| 90780 | IV INFUSION THERAPY; 1 HOUR |
| 90782 | INJECTION; SC/IM |
| 96400 | CHEMOTHERAPY; SC/IM |
| 96402 | CHEMO HORMON ANTINEOPL SQ/IM |
| J1950 | Injection, leuprolide acetate (for depot |
| | suspension), per 3.75 mg |
| J3315 | Injection, triptorelin pamoate, 3.75 mg |
| J9155 | INJECTION, DEGAREUX, 1 MG |
| J9202 | Zoladex ® 3 month |
| J9217 | DepoLupron 1 3 4 month |
| J9218 | Leuprolide acetate, per 1 mg |
| J9219 | Leuprolide acetate implant, 65 mg |
| J9225 | Histrelin implant, 50 mg |

CPT codes are standard codes used in the health industry to identify certain procedures and are consistent across different health records and different medical practices. It should be noted that these CPT codes are for example only and should not be limited to such codes. If, the CPT codes are modified or updated, similar or equivalent CPT codes for the procedures included in Table 1 above could be used in their place.

Once a patient is identified as having a CPT code or a procedure defined as GnRH, the External Patient ID field is populated with the patient's ID, at least one of the one or more Raw Text fields is populated with the procedure name associated with that CPT code and included in the patient's health records, the one or more Procedure Date fields is populated with the procedure date associated with that CPT code or GnRH procedure and the one or more Value fields can be populated, for example, in the following non-limiting manner. In some embodiments, the Value fields, for example, can be populated with: the medication or treatment prescribed/performed on the patient, dosages of any prescribed medications, PSA levels, testosterone values, and the like. The Value fields, therefore, can be populated with data present in the patient's medical health record that is used to calculate the values of other fields. In some embodiments, the CRPC Search Method comprises populating the CRPC Table with PSA levels that have been collected for the identified patient and the associated date of collection for each. These can be populated into the Value fields and analyzed as such.

In some embodiments, the CRPC Search Method can be used to identify other drug names included in the Raw Text fields. That is, the Raw Text fields can be searched by comparing the text in these fields against a list of drugs/medications included in the search method to identify certain drugs that may indicate CRPC. For example, if any of the Raw Text fields in the identified patient's health records includes the word “Vantas,” the Injection field in the CRPC Table is populated with “Vantas.” In some embodiments, drug names are identified in a patient's health records and the Injection field populated with such drug names. In some embodiments, the CRPC Search Method comprises (1) populating the values of the Injection field with the drug names that have been searched and identified in the patient's medical health records and (2) populating the Injection Dose field with the corresponding dose for the drugs populated in the Injection field. In some embodiments, the amount of the drug administered/prescribed can be correlated with a time period. For example, if a patient is administered 45 mg of Lupron (leuprolide), this correlates to a 4 month dose. Therefore, one of skill in the art would understand the length of time the patient was on a certain drug based upon the dose of that drug. Accordingly, the fields can be, in some embodiments, populated based upon the drug and the dose even if certain information is not included in the medical health record.

In some embodiments, the CRPC Search Method comprises populating the CRPC Table with values of testosterone levels that have been collected for the identified patient and the date of collection for each testosterone level. These values can be taken from the Raw Text fields present in the CRPC Table that have been populated from the patient's medical health records for a patient that has been identified as potentially having CRPC.

In some embodiments, the CRPC Search Method comprises interrogating the PSA levels to determine if the patient's PSA levels are rising over a period of time; wherein the interrogation comprises ordering each PSA level by the date it was collected least current to most current, i.e., chronological order. Once the PSA levels are in chronological date order, the CRPC Search Method then compares each successive pair of PSA levels, starting with the least current PSA level, to determine if the least current PSA level, based on the date order, is higher, lower, or equal to the second-least current PSA level wherein:
(1) if the least current PSA level is lower than the second-least current PSA level, the PSA level is rising and the PSA Rise Count field is incremented by one and the PSA Last Trend field is marked as Rising;
(2) if the least current PSA level is higher than the second-least current PSA level, the PSA level is falling and the PSA Fall Count field is incremented by one and the PSA Last Trend field is marked as Falling; or
(3) if the least current PSA level is equal to the second-least current PSA level, the PSA level is not changing and the PSA Last Trend field is marked as Even.
   Next, if the PSA Last Trend field is marked as Rising and the third-least current PSA level is higher than the second-least current PSA level then (a) the PSA Flag field is set to 1, (b) if the First STRO Rise Date field is set to NULL, the First ASTRO Rise Date field is populated with the date (from the one or more Raw Text fields) when the third-least current PSA level was collected from the identified patient and (c) the ASTRO Rise Date field is populated with the date when the third-least current PSA level was collected from the identified patient.

In some embodiments, after the least current, second-least current and third-least current PSA levels are interrogated as outlined above and the associated CRPC Table fields populated or marked accordingly, if there are additional PSA levels to interrogate, the CRPC Search Method interrogates the second-least current PSA level, the third-least current PSA level and the fourth-least current PSA level according to the above steps where the second-least current PSA level now becomes the least-current PSA level, the third-least current PSA level now becomes the second-least current PSA level and the fourth-least current PSA level becomes the third-least current PSA level in the above outlined interrogation. This PSA interrogation process continues, for example, until all consecutive PSA levels have been compared as outlined above. The ASTRO Rise Date Field is only populated with the date of the increase if there are three sequential increases. For example, if the first (least current) PSA level measured is 1 ng/ml, a second (second-least current) PSA level is 2 ng/ml, a third (third-least current or second-most current) PSA level is 2 ng/ml and a fourth (fourth-least current) PSA level is 3 ng/ml, the ASTRO Rise Date field and the First ASTRO Rise Date field would not be populated with the date that the fourth-least current PSA level was taken because the increases were not over three consecutive dates. If, in the example above, the third PSA level had been 2.5 ng/ml, then the ASTRO Rise Date field and the First ASTRO Rise Date field would be populated with the date that the third PSA level was obtained. The date being populated for the ASTRO Rise Date field depends upon an increase in PSA levels. Thus, in some embodiments, a timeline is created where the PSA levels are put in chronological order from least current to most current and then a rise in PSA levels is determined if there is an increase in PSA levels when the PSA levels increase over three consecutive dates when the PSA level was measured. However, to identify the three consecutive dates may require interrogating more than three dates, such as but not limited to 4, 5, 6 or more dates.

Another example of how the First ASTRO Rise Date field and ASTRO Rise Date field are populated based on PSA levels in a patient's medical health records using the above-described interrogation process is included in the chart below.

| | PSA Level | First ASTRO | ASTRO Rise |
|---|---|---|---|
| Date | (ng/ml) | Rise Date | Date |
| Jan. 1, 2012 | 1.00 | Mar. 30, 2012 | NULL |
| Feb. 15, 2012 | 2.00 | Mar. 30, 2012 | NULL |
| Mar. 30, 2012 | 3.00 | Mar. 30, 2012 | Mar. 30, 2012 |
| May 1, 2012 | 2.00 | Mar. 30, 2012 | NULL |
| Jun. 15, 2012 | 1.00 | Mar. 30, 2012 | NULL |
| Aug. 1, 2012 | 3.00 | Mar. 30, 2012 | NULL |
| Sep. 15, 2012 | 4.00 | Mar. 30, 2012 | Sep. 15, 2012 |
| Nov. 1, 2012 | 5.00 | Mar. 30, 2012 | Nov. 1, 2012 |
| Dec. 31, 2012 | 6.00 | Mar. 30, 2012 | Dec. 31, 2012 |

In some embodiments, the CRPC Search Method comprises, setting the CRPC Testosterone field in the CRPC Table to 1 as follows. The CRPC Search Method searches patient medical health records for key words/terms by comparing a database of key words/terms included in the CRPC Search Method to the words/terms included in the medical health records. When key words/terms are found in the medical health records, the one or more Raw Text fields in the CRPC Table for a patient are populated with these key words/terms. One of the terms that the CRPC Search Method searches for is “testosterone.” If “testosterone” is found in a patient's medical health records, one of the Raw Text fields is populated with “testosterone” and a Value field associated with the Raw Text field for “testosterone” is populated with the patient's testosterone level included in the patient's medical health records. Therefore, if a patient has a CRPC Table with a Raw Text field that includes “testosterone” and an associated Value field with a testosterone level less than or equal to 50 ng/ml, the CRPC Testosterone field is set to 1.

In some embodiments, the CRPC Search Method comprises interrogating the values of the Injection and Injection Dose fields to determine if the values of the Injection and Injection Dose fields fall within specified criteria. That is, for example, if the CRPC Table generated for the identified patient includes a value in the ASTRO Rise Date field, and the Injection and the Injection Dose fields fall within specified criteria as described below, then the value of the CRPC No Testosterone field is set to 1. Next, after all of the appropriate records in the CRPC Table that met the above criteria are marked with a CRPC No Testosterone field of 1, the records having a CRPC No Testosterone field of 1 are interrogated again to determine if the patient has a testosterone value less than or equal to 50 ng/ml either 60 days prior to or 60 days after the date included in the ASTRO Rise Date field. If the patient does have this testosterone value, then the CRPC Testosterone field value is set to 1. The Injection and Injection Dose fields fall within specified criteria discussed above if the patient has been treated with:
leuoprolide in an amount of 7.5 mg over a 1 month period within 37 days of the date value in the ASTRO Rise Date field;
leuoprolide in an amount of 40 mg over a 4 month period within 127 days of the date value in the ASTRO Rise Date field;
leuoprolide in an amount of 45 mg over a 6 month period within 187 days of the date value in the ASTRO Rise Date field;
goserelin in an amount of 3.6 mg over a 1 month period within 37 days of the date value in the ASTRO Rise Date field;
goserelin in an amount of 10.8 mg over a 3 month period within 97 days of the date value in the ASTRO Rise Date field;
triptoreline pamoate in an amount of 3.75 mg over a 1 month period within 37 days of the date value in the ASTRO Rise Date field;
triptoreline pamoate in an amount of 11.25 mg over a 3 month period within 97 days of the date value in the ASTRO Rise Date field;
triptoreline pamoate in an amount of 22.5 mg over a 6 month period within 97 days of the date value in the ASTRO Rise Date field;
degarelix acetate in any amount within 37 days of the date value in the ASTRO Rise Date field; or
histrelin acetate in any amount within 372 days of the date value in the ASTRO Rise Date field.

As discussed herein, the date value in the ASTRO Rise Date field is based upon the most-recent date in the most-recent three consecutive PSA level increases after the PSA levels have been placed in date order and interrogated as described herein.

For example, once a table is prepared and the appropriate records are marked as CRPC No Testosterone, i.e., have a CRPC No Testosterone field set to 1, the records are then interrogated again and only the CRPC No Testosterone records where the patient has a qualifying Testosterone value either 60 days prior to or 60 days after the CRPC No Testosterone date, are marked with CRPC Testosterone field equal to 1. An example of how the data can be presented or tabulated is as follows. The data can also be tabulated or presented in a different manner so long as the functions are the same. The names of the fields are for convenience only and any named field could be used so long as it is used to represent the same value and/or information.

[chemical structure]

In this non-limiting example, the records shaded in dark grey have CRPCTesto=0 because the Testosterone measure occurred (ProcedureDate) 100 days after the injection of DepoLupron 1 3 4 month, which corresponds to an Injection value of leuprolide and an Injection Dose value of 4 months. The injection of DepoLupron 1 3 4 month that occurred on Sep. 18, 2013 does not apply because there is no DateASTROrise value on that date, therefore, CRPCNoTesto remains 0. The last 6 rows of the table above have CRPCNoTesto=1 and CRPCTesto=1 because the DepoLupron 1 3 4 month injection occurred on the same day as the qualifying Testosterone measure and the record met the criteria for CRPCNoTesto. Accordingly, the search method and analysis related thereto takes into account the treatment (e.g. medication) in with respect to the other information in the health medical record.

In some embodiments, the patient is identified as having CRPC when the patient has a CRPC No Testosterone field value of 1 or a CRPC Testosterone field value of 1.

As described herein, in some embodiments, methods of identifying a patient with CRPC are provided. In some embodiments, the methods comprise generating a timeline, which can be used to order treatments of a patient where the timeline can include dosage, date, type of medication, name of medication, etc., PSA levels at certain dates, and/or testosterone levels at certain dates. The timeline can be generated by mining data from a patient's medical health record. Once ordered, the timeline is analyzed according to the methods described herein. PSA levels are analyzed to determine if and when there are three consecutive increases in PSA levels as described above and herein. That is, if PSA levels increase over a period of three consecutive dates (a patient may have multiple, three consecutive PSA level increases), the First ASTRO Rise Date field will be set to the most-current date or the last date in the first set of three consecutive PSA level increases and the ASTRO Rise Date field will be set to the same date as the First ASTRO Rise Date field. If there is a second set of three consecutive PSA level increases, the First ASTRO Rise Date field will still be set to the most-current date or the last date in the first set of three consecutive PSA level increases and the ASTRO Rise Date field will then be set to the most-current date or the last date in the second set of three consecutive PSA level increases. That is, the First ASTRO Rise Date will always be set to the most-current date or the last date in the first set of three consecutive PSA level increases and the ASTRO Rise Date field will always be set to the most-current date or the last date in the last or most-recent set of three consecutive PSA level increases. The PSA levels can also be analyzed in conjunction with the drug that a patient has been administered/prescribed. This can also be correlated with the testosterone levels of the patient. This information can be used to populate the fields described above in the CRPC Table and analyzed to determine whether a patient has CRPC. For sake of clarity, the CRPC Table is a non-limiting example of only one table that can be used to determine whether a patient has CRPC. Other tables can be used. Alternatively, the names of the fields are not essential and used solely for ease of reference. Other names or identifiers can be used or those that function in the same manner when the methods described herein are performed.

Methods are also provided herein that can be used to determine if a patient has metastatic prostate cancer. In some embodiments, the Metastatic Prostate Cancer Search Method comprises creating a Patient Metastatic table comprising a set of fields, wherein the set of fields comprises a Patient ID field, an External Patient ID field, a Date field, a HTML field, a Key Word field, a Score field, and a Counts field. Either before or after the Patient Metastatic Table is created, the medical health records database is searched to identify patients that have been diagnosed with secondary malignant neoplasm of bone and bone marrow, which is ICD-9 code 198.5 (where this code is used in a diagnosis field of the patient's medical health records). The Patient Metastatic Table is then populated with data from patients in the medical health records database who have a diagnosis of ICD-9 code 198.5. For these identified patients, the Patient Metastatic Table is populated as follows: the Patient ID field and External Patient ID are populated with the patient information for the identified patient, the Date field is populated with the date of the ICD-9 code 198.5 diagnosis, the HTML field is populated with a value of “198.5,” the Key Word field is populated with the value “Diagnosis,” and the Score field is populated with the value 10000.

It should be noted that the specific ICD-9 code is for example only and should not be limited as such. If, the ICD-9 codes are modified or updated, a similar or equivalent ICD-9 code for secondary malignant neoplasm of bone and bone marrow could be used in place of ICD-9 code 198.5. Accordingly, any inclusion or reference to “198.5” herein or in the Metastatic Prostate Cancer Search Method should not be so limited.

In some embodiments, in order to identify patients that may have not been formally diagnosed with ICD-9 code 198.5 (or any new or updated ICD-9 code that corresponds to secondary malignant neoplasm of bone and bone marrow) or for patients where the ICD-9 code 198.5 is not used in a diagnosis field in their medical health records, the fields in the records/documents in the medical health records database are searched to identify patient documents that contain the phrase “198.5” or the phrase for any new or updated ICD-9 code that corresponds to secondary malignant neoplasm of bone and bone marrow. For such patients, the Patient Metastatic Table is populated as follows: the Patient ID field and External Patient ID are populated with the patient information for the identified patient, the HTML field is populated with the actual HTML text of the office visit, the Key Word field is populated with the phrase “198.5 from PatientOfficeVisitReport” or a similar phrase for any new or updated ICD-9 code that corresponds to secondary malignant neoplasm of bone and bone marrow, and the Score field is populated with the value 10000. The “HTML text” refers to text that is entered into a patient's medical health record by a provider or health care professional. The HTML text could also be referred simply as the text or descriptions contained in the health medical records.

In addition, in some embodiments, in order to identify any patients that could have been missed by the previously disclosed database searches, the medical health records database can be searched to identify patients who have a cancer staging score of M1 or greater. For such patients, the Patient Metastatic Table is populated as follows: the Patient ID field and External Patient ID are populated with the patient information for the identified patient, the HTML field for that patient is not populated, the Key Word field is populated with the phrase “Derived from PatientCancerStaging” and the Score field is populated with the value 1000.

In other embodiments, in order to identify any patients that could have been missed by the previously disclosed database searches, the medical health records database can be searched to identify patients who have specific medications included in their records/data. The specific medications include abiraterone, docetaxel, Taxotere®, Xgeva®, zoledronic, Zometa®, or Zytig®. For such patients, the Patient Metastatic Table can be populated as follows: the Patient ID field and External Patient ID are populated with the patient information for the identified patient, the HTML field is not populated, the Key Word field is populated with the name of the medication(s), and the Score field is populated with the value of 10.

The Patient Metastatic Table can also populated with data for patients who have specific “positive phrases” included in the actual text of the office visits documented in the patient's medical health records. Multiple positive phrases do not lead to the generation of more tables for an individual patient, but rather the table for the patient would be populated with multiple entries. The “positive phrases” can be one of the following: bone mets, bone scan positive, bone scan with metastatic disease, bony metastases, bony metastasis, bony metastatic, bony mets, extensive skeletal metastases, extensive skeletal metastasis, metastatic disease, metastatic prostate cancer, metastatic to bone mets, multiple mets, obvious mets, positive bone scan, positive for bone mets, positive for mets, skeletal metastatic disease, skeletal mets, or widely metastatic prostate cancer. For such patients, the Patient Metastatic Table is populated as follows: the Patient ID field and External Patient ID are populated with the patient information for the identified patient, the HTML field is populated with the actual HTML (text) of the office visit report, the Key Word field is populated with the “positive phrase” from the list above that was found in the patient's medical health records, and the Score field is populated with the value 0.1.

In some embodiments, a single Patient Metastatic Table is created for the medical health records or database that is being searched to identify patients having metastatic prostate cancer. The format of the Patient Metastatic Table can be, for example, a table where the above-discussed fields correspond to columns in the table and the rows correspond to the patients or patient records in the database that have been identified as potentially having metastatic prostate cancer. However, the format of the Patient Metastatic Table is not critical and other formats than those described herein can also be used to arrange the information for analysis.

In some embodiments, in order to remove any records from the Patient Metastatic Table that may have been incorrectly identified a patient as possibly having metastatic prostate cancer based on verbiage included in the text of an office visit, i.e., that were identified based on the medication search or the “positive phrase” search, the actual text of the office visits included in the medical health records for the patients that were identified based on these verbiage searches are searched for specific “negative phrases” used in combination with the above-identified “positive phrases.” That is, the text of the office visits are searched for instances where any of the “negative phrases” listed below precedes any of the “positive phrases” listed above. The “negative phrases” include: appropriate metastatic workup, concern he has prostate cancer metastatic to, did not demonstrate, did not demonstrate any, did not demonstrate any local or distant, did not demonstrate obvious, did not demonstrate obvious osseous, did not demonstrate osseous, did not reveal any, did not show any definite evidence of, did not show any evidence of, did not show any obvious, doubt actual, failed to demonstrate any, failed to show, for bone scan if mets treat, has not been diagnosed with, metastatic disease was not identified, metastatic workup reveals, neg, neg for, negative, negative for, negative for definitive osseous, negative metastatic workup, no, no convincing evidence for, no convincing evidence of, no definite evidence for, no definite evidence of, no definite scintographic evidence of, no evidence for, no evidence of, no evidence to suggest, no gross, no obvious, no obvious evidence of, no radiographic evidence of, no symptoms of, no symptoms or sing of, showed no, or without evidence of. If the Metastatic Prostate Cancer Search Method identifies a Patient Metastatic Table that includes the above-discussed combination of “positive” and “negative” phrases in its HTML field (i.e., the actual office visit text), the Patient Metastatic table is deleted.

It should be noted that the above-described searches to identify patients that (1) have a diagnosis of ICD-9 code 198.5 or similar, (2) include the phrase “198.5” or similar ICD-9 code in their medical health records, (3) have a cancer staging score of M1 or greater, (4) have specific medications listed in their medical health records, or (5) include one of the “positive phrases” discussed above in their medical health records, may result in a patient having multiple “hits” or being identified multiple times as potentially having metastatic prostate cancer. These multiple “hits” result in a patient having multiple entries in his Patient Metastatic Table.

The Counts field is updated with a count of each Key Word field, for each patient, each Date, and each Score. At this point, a Patient Metastatic Table has been created, which includes at least one entry for every patient in the medical health records database that was searched that was identified as possibly having metastatic prostate cancer. As discussed above, a patient can have multiple entries in the Patient Metastatic Table if the patient had multiple “hits” as discussed above.

The Metastatic Prostate Cancer Search Method can also comprise creating a Patient Metastatic Doc Table that contains records for each patient in the Patient Metastatic Table. Each record in the Patient Metastatic Doc Table includes a set of fields comprising an External Patient Id field, a HTML field, a Date field, a Key Word Combined field, a Document Score field, and a Cumulative Score field. Next, for each patient entry in the Patient Metastatic Table, the above-identified fields in the Patient Metastatic Doc Table are populated with the corresponding information from each Patient Metastatic Table entry for that patient and a Document Score and a Cumulative Score value is determined. The Document Score is determined as follows. For each patient, count the number of times a given score occurs according to the following method (i.e., each separate entry in the patient Metastatic table for that patient will result in a “score”): 1) count the number of times the Score 10000 occurs for that patient and record this number as Element1; 2) count the number of times the score 1000 occurs and append the letter m to Element1 if the score 1000 is present; 3) count the number of times the Score 10 occurs and record this number as Element2; and 4) count the number of times the Score 0.1 occurs and record this number as Element3. The Document Score field is then populated in the Patient Metastatic Doc Table for a patient with the derived Document Score in the following format: Element1.Element2.Element3.

For example, if a patient's Patient Metastatic Doc record had one Score of 10000 (Element1=1) and two Scores of 10 (Element2=2) and one Score of 0.1 (Element3=1), the Document Score value would be presented as 1.2.1. In another example, if a patient's Patient Metastatic Doc record had one score of 1000 and one score of 0.1, the Document Score value would be presented as 0m.0.1. The records can be compiled in a table or other type of format.

Once all the Document Score values have been calculated for each entry in the Patient Metastatic Doc Table for each patient, the Cumulative Score value for each patient can be calculated by summing the Document Score Values for the records associated with each patient. For example, a patient has the following Document Score Values, which corresponds to four entries in that patient in the Patient Metastatic Doc Table:
i) 0.1.5
ii) 1.3.1
iii) 1.0.0
iv) 1m.0.2
   The scores are added vertically and the Cumulative Score value for this patient would be presented as 3m.4.8.

A next step in the Metastatic Prostate Cancer Search Method can be to identify and accumulate/combine all of the words/phrases populated in the Key Word fields for each patient included in the Patient Metastatic Doc Table. For example, a patient has the following Key Word values in his Patient Metastatic Doc Table: bone mets, Xgeva. Then the combined Key Word would “bone mets, Xgvea.” This combined Key Word is then populated in the Key Word Combined field of the Patient Metastatic Doc Table.

After the previously discussed steps have been performed, the resulting information/data included in the Patient Metastatic Doc Table is analyzed to assess the likelihood of the patients having metastatic prostate cancer. The patient records in the Patient Metastatic Doc Table with low Cumulative Score values would be removed because these patients would be unlikely to have metastatic prostate cancer. For example, if the records for a patient in the Patient Metastatic Doc Table and hence, that patient, has a Cumulative Score value of 0.1.0, a Key Word value of Zometa and a diagnosis of osteoporosis (e.g. ICD9=733.0), then the patient would be removed. Additionally, a patient who has a Cumulative Score value of 0.0.1 would only have one document (entry) in their medical health records that contains one of the positive phrases defined herein, and thus, would be determined as not likely having metastatic prostate cancer.

A non-limiting example of a table that can be generated is as follows. An example of the Patient Metastatic Docs Table can contain records similar to the following:

| Date | Key Words | Document Score | Cumulative Score |
|---|---|---|---|
| 1900 Jan. 01 | XGeva | 0.1.0 | 11.1.1 |
| 2012 Oct. 11 | mets | 0.0.1 | 11.1.1 |
| 2012 Nov. 13 | Diagnosis | 1.0.0 | 11.1.1 |
| 2012 Dec. 17 | Diagnosis | 1.0.0 | 11.1.1 |
| 2013 Jan. 21 | Diagnosis | 1.0.0 | 11.1.1 |
| 2013 Mar. 04 | Diagnosis | 1.0.0 | 11.1.1 |
| 2013 Apr. 04 | Diagnosis | 1.0.0 | 11.1.1 |
| 2013 May 06 | Diagnosis | 1.0.0 | 11.1.1 |
| 2013 Jun. 10 | Diagnosis | 1.0.0 | 11.1.1 |
| 2013 Aug. 13 | Diagnosis | 1.0.0 | 11.1.1 |
| 2013 Oct. 14 | Diagnosis | 1.0.0 | 11.1.1 |
| 2013 Nov. 12 | Diagnosis | 1.0.0 | 11.1.1 |
| 2013 Dec. 16 | Diagnosis | 1.0.0 | 11.1.1 |

In this non-limiting example, the initial record was based upon a historical medication record that the patient was prescribed XGeva at some point in the past (Document Score 0.1.0), but the date is unknown (1900-01-01). The second record was the result of the word “mets” appearing in an office visit document (Document Score 0.0.1) on 2012-10-11. The remaining scores were the result of a definitive diagnosis of 198.5 somewhere in the patient record (Document Score 1.0.0), resulting in the Cumulative Score of 11.1.1, in this case. Accordingly, the cumulative score is a “sum” of the interrogation of the records. Other medications and keywords can be used as described herein.

The methods of identifying patients with CRPC and metastatic disease disclosed and described herein can be used in conjunction with one another to identify patients with both CRPC and metastatic disease.

The presently described methods may also need information about when a patient was initially diagnosed with prostate cancer. The initial diagnosis date for prostate cancer is determined by the earliest of the following: the diagnosis date of ICD-9 code 198.5 or any new or updated ICD-9 code that corresponds to secondary malignant neoplasm of bone and bone marrow, a definitive treatment event date, a Gleason Score date, a T-Stage value date, a prostate biopsy date prior to a diagnosis of prostate cancer, however, there must be a diagnosis of prostate cancer after the biopsy date for the initial date of the biopsy to be used, or a bone scan date.

Treatments that are considered definitive treatments for prostate cancer are defined as one of the following procedures:

(1) Brachytherapy
(a) CPT: 55875—TRANSPER.I NEEDLE PLACE, PROS
(b) CPT: 77776—APPLY INTERSTIT RADIAT SIMPL
(c) CPT: 77777—APPLY INTERSTIT RADIAT INTER
(d) CPT: 77778—APPLY INTERSTIT RADIAT COMPL
(e) CPT: 77787—HDR BRACHYTX OVER 12 CHAN

(2) Cryotherapy
(a) CPT: 55873—CRYOABLATE PROSTATE
(3) LaparoscopIc Prostatectomy
(a) CPT: 55866—Laparoscopic Radical Retropubic Prostatectomy

(4) PerineaL Prostatectomy
(a) CPT: 55810—EXTENSIVE PROSTATE SURGERY
(b) CPT: 55812—Radical PerineaL Prostatectomy
(c) CPT: 55815—EXTENSIVE PROSTATE SURGERY

(5) Retropubic Prostatectomy
(a) CPT: 55840—EXTENSIVE PROSTATE SURGERY
(b) CPT: 55842 . . . EXTENSIVE PROSTATE SURGERY
(c) CPT: 55845 . . . Radical Retropubic Prostatectomy w/BPLND

(6) Radiation Therapy
(a) CPT: 77401—RADIATION TREATMENT DELIVERY
(b) CPT: 77402—RADIATION TREATMENT DELIVERY
(c) CPT: 77403—RADIATION TREATMENT DELIVERY
(d) CPT: 77404—RADIATION TREATMENT DELIVERY
(e) CPT: 77406—RADIATION TREATMENT DELIVERY
(f) CPT: 77407—RADIATION TREATMENT DELIVERY
(g) CPT: 77408—RADIATION TREATMENT DELIVERY
(h) CPT: 77409—RADIATION TREATMENT DELIVERY
(i) CPT: 77411—RADIATION TREATMENT DELIVERY
(j) CPT: 77412—RADIATION TREATMENT DELIVERY
(k) CPT: 77413—RADIATION TREATMENT DELIVERY
(l) CPT: 77416—RADIATION TREATMENT DELIVERY
(m) CPT: 77417—RADIOLOGY PORT FILM(S)
(n) CPT: 77418—RADIATION TX DELIVERY; IMRT
(o) CPT: 77421—STEREOSCOPIC X-RAY GUIDANCE
(p) CPT: 77525—PROTON TREATMENT; COMPLEX
(q) CPT: 77427—RADIATION TX MANAGEMENT; X5.

As previously discussed, these CPT codes are for example only and should not be limited to such codes. If the CPT codes are modified or updated, similar or equivalent CPT codes for the procedures set forth above could be used in their place.

In some embodiments, the methods comprise confirmation of the diagnosis of CRPC status, for example, by a doctor. Once confirmed, a course of treatment can be prescribed. Examples of CRPC treatments include, and are not limited to, administering a CYP17 inhibitor or an androgen receptor antagonist. The cytochrome P450 (CYP) enzymes include a large family of highly conserved enzymes, including CYP17, that are involved in the synthesis of cholesterol and other bioactive steroids. The fact that these enzymes are involved in steroid hormone biosynthesis has led to recent findings that CRPC in men and certain breast cancers in women are responsive to CYP17 inhibition. Inhibitors of CYP17 or other compounds, which antagonize the androgen receptors on prostate tumor cells can be used to treat CRPC.

Ketoconazole has been used to inhibit CYP17, but is not very potent and is non-selective because it inhibits other CYP enzymes. Other CYP17 inhibitors have been reported, and the steroidal CYP 17 inhibitor Zytiga® (abiraterone acetate) was recently approved by the U.S. Food and Drug Administration (FDA) for use in combination with prednisone for the treatment of patients with metastatic castration-resistant prostate cancer who have received prior chemotherapy containing docetaxel.

Other compounds suitable for treating CRPC are known in the art, and include docetaxel, TAK-700 (Takeda Pharmaceuticals), MDV3100 (Medivation) and azoyl steroids such as 17-(5′-isoxazolyl)androsta-4,16-dien-3-one (L-39) (Grigoryev D N, Long B J, Nnane I P, Njar V C, Liu Y, Brodie A M, Br. J. Cancer., 81, 622-630, 1999; Nnane I P, Long B J, Ling Y Z, Grigoryev D N, Brodie A M, Br. J. Cancer 83: 74-82, 2000). Other non-steroidal androgen receptor antagonists suitable for treating CRPC have been described; e.g., in EP 100172, EP 1790640, U.S. Pat. No. 6,087,509, U.S. Pat. No. 6,673,799, U.S. Pat. No. 7,271,188, WO 03/057669, WO 2004/099188, WO 2006/133567, WO 2008/124000, WO 2009/028543, WO 2009/055053 and WO 2011/051540.

### EXAMPLES

### Example 1

The CRPC Search Method disclosed herein, was applied to a medical health records database that included 705 patients. The CRPC Search Method successfully identified 114 patients with CRPC. No patients were falsely (positively) identified as having CRPC. In addition, 100 additional patients were reviewed and no patients were falsely identified as not having CRPC (no false negatives). Inclusion of the testosterone criteria limited the number of patients detected, but did not impact the accuracy. The reviewers concluded that inclusion of testosterone criteria is not practically necessary, but should it be required, it appears it would function accurately.

It is to be understood that the embodiments disclosed herein are not limited to the specific devices, methods, conditions, or parameters described and/or shown herein, and that the terminology used herein is for the purpose of describing particular embodiments by way of example only. Thus, the terminology is intended to be broadly construed and is not intended to be limiting. For example, as used in the specification including the appended numbered paragraphs, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

Although the present embodiments have been described above in exemplary terms, the embodiments are not limited thereto. Rather, the appended numbered paragraphs should be construed broadly to include other variants and embodiments which may be made by those skilled in the art without departing from the scope and range of equivalents of the embodiments.

What is claimed is:

1. A method of identifying a patient with castrate resistant prostate cancer (“CRPC”) comprising:
i) creating a table with fields comprising:
a) an External Patient ID field;
b) one or more Raw Text fields;
c) one or more Procedure Date fields;
d) one or more Value fields;
e) one or more Injection fields;
f) one or more Injection Dose fields;
g) a Testosterone Flag field set to zero;
h) a PSA Flag field set to zero;
i) a First ASTRO Rise date field;
j) a ASTRO Rise date field,
k) a PSA Rise Count field set to zero;
l) a PSA Fall Count filed set to zero;
m) a PSA Last Trend field
n) a CRPC No Testosterone field set to zero; and
o) a CRPC Testosterone field set to zero;
ii) populating the External Patient ID field, the one or more Raw Text fields, the one or more Procedure Date fields and the one or more Value fields with data from a patient's medical health records, wherein the patient's medical health records include a procedure defined as gonadotrophin-releasing hormone therapy;
iii) populating the table with PSA levels that have been collected for the patient and the date of collection;
iv) populating the values of the Injection field and the Injection Dose field;
v) populating the table with values of testosterone that have been collected for the patient and the date of collection;
vi) interrogating the PSA levels to determine if the PSA levels are rising, wherein the interrogation comprises:
a) ordering each PSA level by the date it was collected from least current to most current;
b) determining if the least current PSA level is lower than a second-least current PSA level, wherein if the least current PSA level is lower than the second-least current PSA level, determining if the second-least current PSA level is lower than the third-least current PSA level:
wherein (1) if the least current PSA level is lower than the second-least current PSA level and the second-least current PSA level is lower than the third-least current PSA level and (2) if the First ASTRO Rise Date field is NULL, populating the First ASTRO Rise Date field with the date that the third-least current PSA level was collected and,
wherein if the least current PSA level is lower than the second-least current PSA level and the second-least current PSA level is lower than the third-least current PSA level, populating the ASTRO Rise Date field with the date that the third-least current PSA level was collected;
c) determining if PSA levels exist that are more current than the third-least current PSA level, wherein if PSA levels exist that are more current than the third-least current PSA level, repeating step (d) for each PSA level that exists that is more current than the third-least current PSA level;
vii) reviewing the patient's medical health records for the term “testosterone,” wherein if “testosterone” is found, populating the one or more Raw Text fields with the term “testosterone” and populating a Value field associated with the term “testosterone” with a testosterone level included in the patient's medical health records;
viii) populating the Testosterone Flag field with a value of 1 when the one or more Raw Text fields includes the term “testosterone” and the testosterone level is less than or equal to 50 ng/ml;
ix) interrogating the values of the Injection and Injection Dose fields to determine if the values of the Injection and Injection Dose fields fall within specified criteria, wherein the specified criteria is met when the patient has been treated with:
leuoprolide in an amount of 7.5 mg over 1 month within 37 days of the date value in the ASTRO Rise Date field;
leuoprolide in an amount of 40 mg over 4 months within 127 days of the date value in the ASTRO Rise Date field;
leuoprolide in an amount of 45 mg over 6 months within 187 days of the date value in the ASTRO Rise Date field;
goserelin in an amount of 3.6 mg over 1 month within 37 days of the date value in the ASTRO Rise Date field;
goserelin in an amount of 10.8 mg over 3 months within 97 days of the date value in the ASTRO Rise Date field;
triptoreline pamoate in an amount of 3.75 mg over 1 month within 37 days of the date value in the ASTRO Rise Date field;
triptoreline pamoate in an amount of 11.25 mg over 3 months within 97 days of the date value in the ASTRO Rise Date field;
triptoreline pamoate in an amount of 22.5 mg over 6 months within 97 days of the date value in the ASTRO Rise Date field;
degarelix acetate in any amount within 37 days of the date value in the ASTRO Rise Date field; or
histrelin acetate in any amount within 372 days of the date value in the ASTRO Rise Date field;
wherein if there is a date value in the ASTRO Rise Date field and if the specified criteria for the Injection and Injection Dose fields is met, populating the CRPC No Testosterone field with a value of 1;
x) determining if the CRPC No Testosterone field is populated with a value of 1, wherein if the CRPC No Testosterone field is populated with a value of 1, interrogating the testosterone level to determine if the testosterone level is less than or equal to 50 ng/ml 60 days prior to the date value in the ASTRO Rise Date field or 60 days after the date value in the ASTRO Rise Date field, wherein if the patient does have a testosterone level that is less than or equal to 50 ng/ml 60 days prior to the date value in the ASTRO Rise Date field or 60 days after the date value in the ASTRO Rise Date field, populating the CRPC Testosterone field with a value of 1; and
xi) identifying the patient as having CRPC when the table has a CRPC No Testosterone field value of 1 or a CRPC Testosterone field value of 1.

2. A method of treating a patient with castrate resistant prostate cancer (“CRPC”), the method comprising:
A) identifying a patient as having CRPC, wherein identifying the patient as having CRPC comprises:
i) creating a table with fields comprising:
a) an External Patient ID field;
b) one or more Raw Text fields;
c) one or more Procedure Date fields;
d) one or more Value fields;
e) one or more Injection fields;
f) one or more Injection Dose fields;
g) a Testosterone Flag field set to zero;
h) a PSA Flag field set to zero;
i) a First ASTRO Rise date field;
j) a ASTRO Rise date field,
k) a PSA Rise Count field set to zero;
l) a PSA Fall Count filed set to zero;
m) a PSA Last Trend field
n) a CRPC No Testosterone field set to zero; and
o) a CRPC Testosterone field set to zero;
ii) populating the External Patient ID field, the one or more Raw Text fields, the one or more Procedure Date fields and the one or more Value fields with data from the patient's medical health records, wherein a patient's medical health records include a procedure defined as gonadotrophin-releasing hormone therapy;
iii) populating the table with PSA levels that have been collected for the patient and the date of collection;
iv) populating the values of the Injection field and the Injection Dose field;
v) populating the table with values of testosterone that have been collected for the patient and the date of collection;
vi) interrogating the PSA levels to determine if the PSA levels are rising, wherein the interrogation comprises:
d) ordering each PSA level by the date it was collected from least current to most current;
e) determining if the least current PSA level is lower than a second-least current PSA level, wherein if the least current PSA level is lower than the second-least current PSA level, determining if the second-least current PSA level is lower than the third-least current PSA level:
wherein (1) if the least current PSA level is lower than the second-least current PSA level and the second-least current PSA level is lower than the third-least current PSA level and (2) if the First ASTRO Rise Date field is NULL, populating the First ASTRO Rise Date field with the date that the third-least current PSA level was collected and,
wherein if the least current PSA level is lower than the second-least current PSA level and the second-least current PSA level is lower than the third-least current PSA level, populating the ASTRO Rise Date field with the date that the third-least current PSA level was collected;
f) determining if PSA levels exist that are more current than the third-least current PSA level, wherein if PSA levels exist that are more current than the third-least current PSA level, repeating step (d) for each PSA level that exists that is more current than the third-least current PSA level;
vii) reviewing the patient's medical health records for the term “testosterone,” wherein if “testosterone” is found, populating the one or more Raw Text fields with the term “testosterone” and populating a Value field associated with the term “testosterone” with a testosterone level included in the patient's medical health records;
viii) populating the Testosterone Flag field with a value of 1 when the one or more Raw Text fields includes the term “testosterone” and the testosterone level is less than or equal to 50 ng/ml;
ix) interrogating the values of the Injection and Injection Dose fields to determine if the values of the Injection and Injection Dose fields fall within specified criteria, wherein the specified criteria is met when the patient has been treated with:
leuoprolide in an amount of 7.5 mg over 1 month within 37 days of the date value in the ASTRO Rise Date field;
leuoprolide in an amount of 40 mg over 4 months within 127 days of the date value in the ASTRO Rise Date field;
leuoprolide in an amount of 45 mg over 6 months within 187 days of the date value in the ASTRO Rise Date field;
goserelin in an amount of 3.6 mg over 1 month within 37 days of the date value in the ASTRO Rise Date field;
goserelin in an amount of 10.8 mg over 3 months within 97 days of the date value in the ASTRO Rise Date field;
triptoreline pamoate in an amount of 3.75 mg over 1 month within 37 days of the date value in the ASTRO Rise Date field;
triptoreline pamoate in an amount of 11.25 mg over 3 months within 97 days of the date value in the ASTRO Rise Date field;
triptoreline pamoate in an amount of 22.5 mg over 6 months within 97 days of the date value in the ASTRO Rise Date field;
degarelix acetate in any amount within 37 days of the date value in the ASTRO Rise Date field; or
histrelin acetate in any amount within 372 days of the date value in the ASTRO Rise Date field;
wherein if there is a date value in the ASTRO Rise Date field and if the specified criteria for the Injection and Injection Dose fields is met, populating the CRPC No Testosterone field with a value of 1;
x) determining if the CRPC No Testosterone field is populated with a value of 1, wherein if the CRPC No Testosterone field is populated with a value of 1, interrogating the testosterone level to determine if the testosterone level is less than or equal to 50 ng/ml 60 days prior to the date value in the ASTRO Rise Date field or 60 days after the date value in the ASTRO Rise Date field, wherein if the patient does have a testosterone level that is less than or equal to 50 ng/ml 60 days prior to the date value in the ASTRO Rise Date field or 60 days after the date value in the ASTRO Rise Date field, populating the CRPC Testosterone field with a value of 1; and
xi) identifying the patient as having CRPC when the table has a CRPC No Testosterone field value of 1 or a CRPC Testosterone field value of 1; and
B) treating the patient identified as having CRPC;

3. The method of claim 2, wherein a treatment for treating the patient identified as having CRPC is selected from the group consisting of administering a CYP17 inhibitor, administering an androgen receptor modulator, administering a cytotoxin, performing a partial prostatectomy, performing a full prostatectomy, and ablating prostate cancer tissue or cells.

4. The method of claim 3, wherein ablating the prostate cancer tissue or cells comprises freezing.

5. A data processing system for identifying a patient with castrate resistant prostate cancer (“CRPC”), the data processing system comprising a computer processor for processing data, wherein the computer processor is configured to (A) accesses an electronic medical health records database and (B) execute a search method on data included in the electronic medical health records database, wherein the search method comprises:
i) creating a table with fields comprising:
a) an External Patient ID field;
b) one or more Raw Text fields;
c) one or more Procedure Date fields;
d) one or more Value fields;
e) one or more Injection fields;
f) one or more Injection Dose fields;
g) a Testosterone Flag field set to zero;
h) a PSA Flag field set to zero;
i) a First ASTRO Rise date field;
j) a ASTRO Rise date field,
k) a PSA Rise Count field set to zero;
l) a PSA Fall Count filed set to zero;
m) a PSA Last Trend field
n) a CRPC No Testosterone field set to zero; and
o) a CRPC Testosterone field set to zero;
ii) populating the External Patient ID field, the one or more Raw Text fields, the one or more Procedure Date fields and the one or more Value fields with data from a patient's medical health records, wherein the patient's medical health records include a procedure defined as gonadotrophin-releasing hormone therapy;
iii) populating the table with PSA levels that have been collected for the patient and the date of collection;
iv) populating the values of the Injection field and the Injection Dose field;
v) populating the table with values of testosterone that have been collected for the patient and the date of collection;
vi) interrogating the PSA levels to determine if the PSA levels are rising, wherein the interrogation comprises:
g) ordering each PSA level by the date it was collected from least current to most current;
h) determining if the least current PSA level is lower than a second-least current PSA level, wherein if the least current PSA level is lower than the second-least current PSA level, determining if the second-least current PSA level is lower than the third-least current PSA level:
wherein (1) if the least current PSA level is lower than the second-least current PSA level and the second-least current PSA level is lower than the third-least current PSA level and (2) if the First ASTRO Rise Date field is NULL, populating the First ASTRO Rise Date field with the date that the third-least current PSA level was collected and,
wherein if the least current PSA level is lower than the second-least current PSA level and the second-least current PSA level is lower than the third-least current PSA level, populating the ASTRO Rise Date field with the date that the third-least current PSA level was collected;
i) determining if PSA levels exist that are more current than the third-least current PSA level, wherein if PSA levels exist that are more current than the third-least current PSA level, repeating step (d) for each PSA level that exists that is more current than the third-least current PSA level;
vii) reviewing the patient's medical health records for the term “testosterone,” wherein if “testosterone” is found, populating the one or more Raw Text fields with the term “testosterone” and populating a Value field associated with the term “testosterone” with a testosterone level included in the patient's medical health records;
viii) populating the Testosterone Flag field with a value of 1 when the one or more Raw Text fields includes the term “testosterone” and the testosterone level is less than or equal to 50 ng/ml;
ix) interrogating the values of the Injection and Injection Dose fields to determine if the values of the Injection and Injection Dose fields fall within specified criteria, wherein the specified criteria is met when the patient has been treated with:
leuoprolide in an amount of 7.5 mg over 1 month within 37 days of the date value in the ASTRO Rise Date field;
leuoprolide in an amount of 40 mg over 4 months within 127 days of the date value in the ASTRO Rise Date field;
leuoprolide in an amount of 45 mg over 6 months within 187 days of the date value in the ASTRO Rise Date field;
goserelin in an amount of 3.6 mg over 1 month within 37 days of the date value in the ASTRO Rise Date field;
goserelin in an amount of 10.8 mg over 3 months within 97 days of the date value in the ASTRO Rise Date field;
triptoreline pamoate in an amount of 3.75 mg over 1 month within 37 days of the date value in the ASTRO Rise Date field;
triptoreline pamoate in an amount of 11.25 mg over 3 months within 97 days of the date value in the ASTRO Rise Date field;
triptoreline pamoate in an amount of 22.5 mg over 6 months within 97 days of the date value in the ASTRO Rise Date field;
degarelix acetate in any amount within 37 days of the date value in the ASTRO Rise Date field; or
histrelin acetate in any amount within 372 days of the date value in the ASTRO Rise Date field;
wherein if there is a date value in the ASTRO Rise Date field and if the specified criteria for the Injection and Injection Dose fields is met, populating the CRPC No Testosterone field with a value of 1;
x) determining if the CRPC No Testosterone field is populated with a value of 1, wherein if the CRPC No Testosterone field is populated with a value of 1, interrogating the testosterone level to determine if the testosterone level is less than or equal to 50 ng/ml 60 days prior to the date value in the ASTRO Rise Date field or 60 days after the date value in the ASTRO Rise Date field, wherein if the patient does have a testosterone level that is less than or equal to 50 ng/ml 60 days prior to the date value in the ASTRO Rise Date field or 60 days after the date value in the ASTRO Rise Date field, populating the CRPC Testosterone field with a value of 1; and
xi) identifying the patient as having CRPC when the table has a CRPC No Testosterone field value of 1 or a CRPC Testosterone field value of 1.
